(19) United States
(12) Patent Application Publication
(10) Pub. No.: US 2021/0196340 A1
(43) Pub. Date: Jul. 1, 2021

(54) COLD PLASMA GENERATING DEVICE WITH POSITIONAL CONTROL

(71) Applicant: L'OREAL, Paris (FR)

(72) Inventor: Kyle Yeates, Redmond, WA (US)

(73) Assignee: L'OREAL, Paris (FR)

(21) Appl. No.: 17/138,636

(22) Filed: Dec. 30, 2020

Related U.S. Application Data
(60) Provisional application No. 62/955,022, filed on Dec. 30, 2019.

(51) Int. Cl. A61B 18/04; A61B 18/10; A61B 18/08

(52) U.S. Cl. CPC A61B 18/042; A61B 2018/00452; A61B 18/082; A61B 18/10

(57) ABSTRACT

A cold plasma device is suitable for treating a region of a biological surface. The device includes a cold plasma generator with an electrode and a dielectric barrier. The dielectric barrier includes a first side that faces the electrode and a second side that faces away from the electrode. An actuator is configured to selectively reciprocate the cold plasma generator between a first position and a second position, and a controller is programmed to control the actuator to selectively position the cold plasma generator relative to the biological surface.

### CROSS-REFERENCE TO RELATED APPLICATION

This application claims the benefit of Provisional Application No. 62/955,022, filed Dec. 30, 2019, the entire disclosure of which is hereby incorporated by reference herein.

### SUMMARY

The application of a cold atmospheric plasma (also referred to as “cold plasma” or “plasma”) to biological surfaces introduces challenges for skin treatment, arising from the complex biological system interactions. In practice, surface conditions and plasma parameters are coupled, where variation in one induces changes in the other. A sudden shift in surface moisture, for example, may affect electrical conductivity of the surface and lead to an increase in plasma intensity. Conversely, a sudden increase in plasma intensity may vaporize moisture from the surface, in turn changing the properties of plasma. This variability and multi-parameter coupling necessitates control of the plasma treatment device.

Complex interactions between light emission from the plasma, plasma generated species, and biological chemicals native to biological surfaces further complicates cold plasma therapy. In some cases, plasma generated species may acidify a biological surface, thereby aggravating preexisting conditions and outweighing any beneficial outcomes of plasma treatment, for example by light emission, or by exposure to plasma generated species that stimulate wound healing or that would otherwise denature harmful bacteria present in the biological surface.

In some applications, generating the cold plasma away from the biological surface (e.g., skin) may be advantageous in comparison to generating the cold plasma proximately to the biological surface. When the cold plasma is generated away from the biological surface, the concentration, temperature, pressure, and other properties of the plasma can be controlled less tightly than when the plasma is generated directly at the biological surface. For example, whereas the temperature of the air that carries the cold plasma toward the biological surface has to be within a relatively narrow range (to avoid discomfort to the user), the range of temperatures for the incoming air is wider when the plasma is generated away from the biological surface. Subsequent to generating the cold plasma, the temperature of the air may be lowered or raised to a more acceptable range while the plasma is still within the cold plasma generating device. In some embodiments, the concentration of the plasma species may also be higher for the plasma generated away from the biological surface, because the concentration of the plasma species can be reduced inside the device before the cold plasma reaches the biological surface. For example, the concentration of the plasma species and the temperature of the air will generally decrease with time elapsed from the creation of the plasma species.

### Cold Plasma Therapy Devices

Non-thermal “cold” atmospheric plasma can interact with living tissue and cells during therapeutic treatment in multiple ways. Among the possible applications, cold atmospheric plasma may be used in biology and medicine for sterilization, disinfection, decontamination, and plasma-mediated wound healing.

Several commercialized devices are certified for medical treatment at the present time. These devices are not designed for home use by consumers. Instead, they are designed for use by medical technicians with expertise and training in medical treatment techniques. An example of such device is Rhytec Portrait®, which is a plasma jet tool for topical dermatological treatments. This device features complex power supplies with tightly regulated parameters, using radio-frequency power sources. In addition, the Bovie J-Plasma®, the Canady Helios Cold Plasma, and the Hybrid Plasma™ Scalpel are all available for use as medical treatment devices. In Germany, the kINPen®, also a plasma jet device, and the PlasmaDerm®, a dielectric barrier discharge (DBD) device, are both certified medical devices that have been introduced to the market within recent years. These devices aim at medical treatment of human tissues, either externally, as in the PlasmaDerm®, or internally. In contrast with the plasma devices for the medical use, the devices for the cosmetic use are geared for a generally intuitive use by consumers, resulting in cosmetic care and pleasant sensation, as opposed to well controlled and certifiable therapeutic effect.

FIG. 1 is a schematic diagram of a plasma generator 10 in accordance with prior art. As shown in FIG. 1, a cold plasma 18 forms through disparate excitation of electrons in a plasma gas by electric fields, relative to the milder excitation effect of the fields on the more massive nuclei of the plasma gas. The cold plasma 18 is formed between a live electrode 14 and a ground electrode 15, also called a counter-electrode, when the live electrode 14 is energized relative to the ground electrode 15 by a power source 12. The power source 12 is an alternating current source or an amplitude modulated direct current source. The cold plasma 18 is a dielectric barrier discharge if the plasma generator 10 includes a dielectric barrier 16 that is placed against the live electrode 14. The cold plasma 18 contains both high temperature electrons 19 and low temperature ions 19 and neutral species. In conventional systems, the plasma gas includes noble gases like helium or argon, and also oxygen and nitrogen containing gases to form reactive oxygen and nitrogen species (RONS). In some cases, as with the PlasmaDerm®, the plasma forms directly in air.

FIG. 2 is an image of dielectric barrier discharges 20 in operation in accordance with prior art. FIG. 2 was obtained as a plan view through a transparent electrode. The plasma 18 forms as multiple discrete filamentary discharges that individually form conductive bridges for ions and electrons 19 to migrate between the electrodes.

For topical treatment, several forms of plasma are used. The first is the gas jet plasma that provides a jet of ions and reactive species that can be directed to a target over varying distances, typically at distances greater than a few millimeters. The medical plasmas described in a preceding paragraph typically feature a gas jet plasma. A second form is the Floating Electrode Dielectric Barrier Discharge (FE-DBD) devices, in which the target substrate (often the human body) acts as a floating ground electrode. The third form is a DBD plasma wand, where the dielectric barrier is placed against a floating ground, instead of the live electrode, and may take the form of a fluorescent tube. The fourth form is a coordinated plurality of dielectric barrier discharge sources. In such an arrangement, a number of atmospheric FE-DBD plasma sources are incorporated into a handheld or flexible device, that is then used to treat one or more anatomical regions.

FIGS. 3A and 3B are two views of a cold plasma device in accordance with prior art. A skin treatment device 30 produces cold plasma 18 through a unitary structure that includes a head 31 and a body 34. The device includes one or more user controls, including a plasma power switch 32, and a light switch 33. The head 31 includes one or more light emitting diodes 35 (LEDs). The skin treatment device 30 further includes a plasma pulse control 37, configured to create the plasma 18 at the head 31 while the plasma pulse control 37 is pressed. The skin treatment device 30 includes a charging port 36 for charging an enclosed battery. The skin treatment device 30 includes internal electronic components that drive the plasma 18.

FIG. 4 is a block diagram of a cold plasma device in accordance with prior art. Electronic components 40 include a unitary structure having a DBD head 47 and body 42. The cold plasma 18 is produced between electrodes included in the DBD head 47, which serves as the treatment site. The DBD head 47 is electrically connected to a high voltage unit 45, providing power to the DBD head 47. The power needed to drive the plasma 18 is provided by a rechargeable battery pack 43 enclosed within the body 42. The system includes one or more LEDs 46, connected to the system through a main PC board and control circuitry 44. The main PC board and control circuitry 44 controls the flow of electricity to the LED 46 and the high voltage unit 45 and receives input from one or more user controls 48 and external power in 49 to charge the rechargeable battery pack 43.

Without being bound to theory, it is believed that the effect of cold atmospheric plasma therapy is due to some extent to interaction between RONS and biological systems. A non-exhaustive list of RONS includes: hydroxyl (OH), atomic oxygen (O), singlet delta oxygen (O₂(¹Δ)), superoxide (O₂⁻), hydrogen peroxide (H₂O₂), and nitric oxide (NO). Hydroxyl radical attack is believed to result in peroxidation of cell membrane lipids, in turn affecting cell-cell interaction, regulation of membrane-protein expression, and many other cellular processes. Hydrogen peroxide is a strong oxidizer, believed to have a harmful effect on biological systems. Nitric oxide is believed to play a role in cell-cell signaling and bio-regulation. At the cellular level, nitric oxide is believed to affect regulation of immune deficiencies, cell proliferation, phagocytosis, collagen synthesis, and angiogenesis. At the system level, nitric oxide is a potent vasodilator.

Cold atmospheric plasmas also expose biological surfaces to electric fields, on the order of 1-10 kV/cm. It is believed that cells respond to such fields by opening trans-membrane pores. Such electric-field induced cellular electroporation is believed to play a role in transfusion of molecules across cell membranes. Without being bound to theory, the efficacy of treatment is believed to be due at least in part to long-lived plasma-generated species, which in an air plasma will be a variety of RONS at concentrations particular to the operating parameters of the cold atmospheric plasma source.

While cold atmospheric plasma can also be used to ablate tissue or effect treatment in a very short time when operated at high power and intensity, such treatment is believed to harm surrounding tissue and to penetrate far beyond the treated area. Without being bound to theory, it is believed that cold atmospheric plasma treatment at low intensity avoids damaging cells.

Without being bound to theory, it is believed that an important parameter both for direct cold atmospheric plasma treatment and for indirect treatment using plasma-treated media is the dose of plasma species imparted to the treatment surface. In general, this is expressed as a concentration of a given plasma species produced by the cold atmospheric plasma source that is imparted to a unit area of the treated surface over a unit time.

Alternatively, the dose may be expressed as a simple length of time, if the treatment has been determined and the behavior of the cold atmospheric plasma source is well understood. For example, for a stable cold atmospheric plasma source and a uniform surface, a particular dose of a given RONS will be achieved after the cold atmospheric plasma has treated the uniform surface for a given length of time. In practice, surface conditions and plasma characteristics are coupled, where variation in one induces changes in the other. A sudden shift in surface moisture, for example, may affect the conductivity of the surface and lead to an increase in plasma intensity. Conversely, a sudden increase in plasma intensity may vaporize moisture from the surface, producing RONS and changes in the surface. This variability necessitates control of the plasma treatment device, as discussed in greater detail below.

Without being bound to theory, it is believed that cold atmospheric plasma treatment penetrates into the treatment surface through a synergistic effect of electroporation, permeability of plasma generated species, and cell-to-cell signaling. The so called “bystander effect” is thought to play a role in propagating plasma induced cellular changes away from the treatment surface and into a volume beneath it. The bystander effect is believed to occur through chemical signals passed between cells in response to the introduction of a biologically active chemical, potentially amplifying the magnitude of the treatment impact.

In experiments it has been shown that RONS include reactive nitrogen species (RNS) and reactive oxygen species (ROS) that are believed to interact in differing ways to diverse biological surfaces. In agarose films, for example, RONS permeate a volume beneath the film, while in living tissues, only RNS will do so. ROS do penetrate, however, into gelatin and other liquids. ROS, being more reactive than RNS are shorter-lived and are believed to be linked in some circumstances to aggressive or harmful effects on biological surfaces, as previously discussed with respect to hydrogen peroxide.

### Cold Plasma Generating Device with Positional Control

An embodiment of cold plasma device is suitable for treating a region of a biological surface. The device includes a cold plasma generator having an electrode and a dielectric barrier. The dielectric barrier has a first side that faces the electrode and a second side that faces away from the electrode. An actuator is configured to selectively reciprocate the cold plasma generator between a first position and a second position. The cold plasma device further includes a controller operably coupled to the actuator and programmed to control the actuator to selectively position the cold plasma generator relative to the biological surface.

In an embodiment, the cold plasma device further includes a sensor configured to sense a distance between the cold plasma generator and the biological surface.

In an embodiment, the controller is configured to control the actuator to maintain the cold plasma generator at a predetermined distance from the biological surface.

In an embodiment, the controller is configured to receive a signal from the sensor, and the controller is programmed to control the actuator according to the received signal.

In an embodiment, the sensor senses a distance between the cold plasma generator and the biological surface.

In an embodiment, the sensor senses a feature on the biological surface, and the controller is programmed to control the actuator to position the cold plasma generator according to the sensed feature.

In an embodiment, the actuator is a linear actuator.

In an embodiment, the actuator is a rotary actuator.

In an embodiment, the cold plasma device further includes a housing, and the cold plasma generator is moveably mounted to the housing.

In an embodiment, the cold plasma generator is slidably mounted to the housing

An embodiment of a disclosed method treats a biological surface with a cold plasma generator that is located a distance from the biological surface and generates a plasma concentration. The method includes sensing the distance between the cold plasma generator and the biological surface and comparing the sensed distance to a predetermined distance. The method further includes moving the cold plasma generator relative to the biological surface according to the comparison of the sensed distance and the predetermined distance.

In an embodiment, the predetermined distance is a target distance, and the step of moving the cold plasma generator moves the cold plasma generator such that the sensed distance approaches the predetermined distance.

In an embodiment, the method further includes the step of sensing a feature on the biological surface.

In an embodiment, the method further includes the step of adjusting the plasma concentration generated by the cold plasma generator according to the sensed feature.

In an embodiment, the plasma concentration generated is adjusted according to the sensed feature and the sensed distance.

In an embodiment, the method further includes the step of adjusting the distance between the cold plasma generator and the biological surface according to the sensed feature.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

The foregoing aspects and many of the attendant advantages of this invention will become more readily appreciated as the same become better understood by reference to the following detailed description, when taken in conjunction with the accompanying drawings, wherein:

FIG. 1 is a schematic diagram of a plasma generator in accordance with prior art;

FIG. 2 is an image of a dielectric barrier discharge surface in operation in accordance with prior art;

FIGS. 3A-3B are two views of a cold plasma device in accordance with prior art;

FIG. 4 is a block diagram of a cold plasma device in accordance with prior art;

FIG. 5 is a partial side view of a representative embodiment of a cold plasma device in accordance with the present disclosure;

FIG. 6 is a schematic diagram thereof;

FIG. 7 is a partial side view thereof.

FIG. 8 is a flowchart of an embodiment of a method for treating a biological surface with a cold plasma generator according to the present disclosure; and

FIG. 9 is a flowchart of an embodiment of a method for treating a biological surface with a cold plasma generator according to the present disclosure.

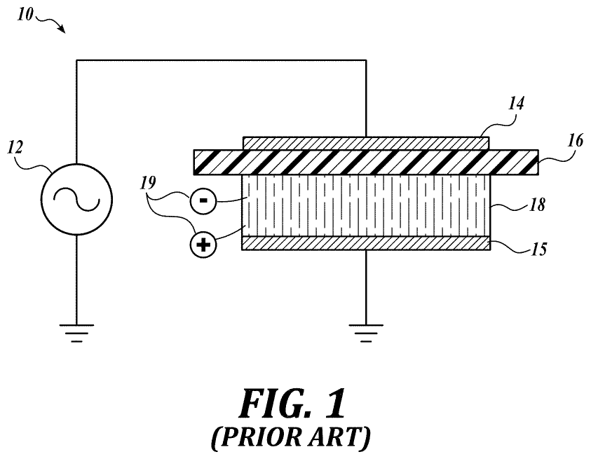

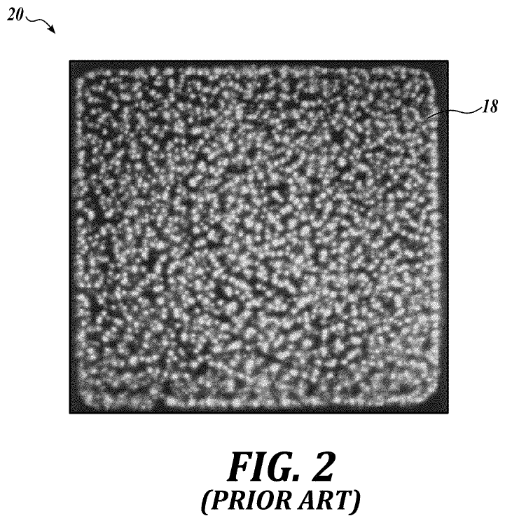

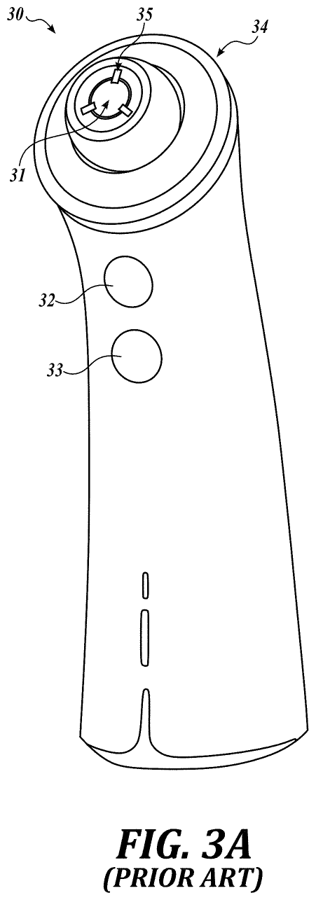

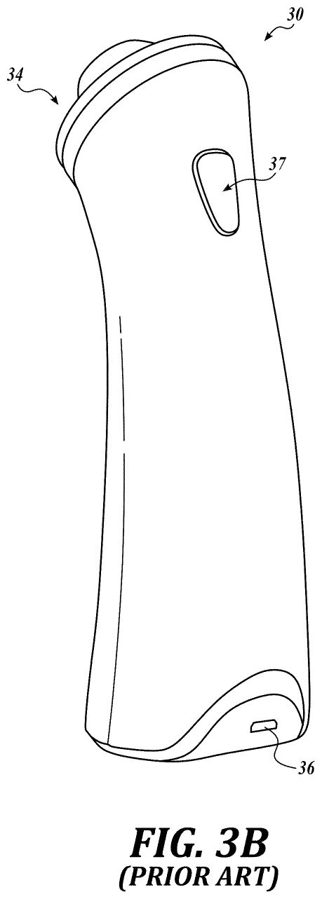

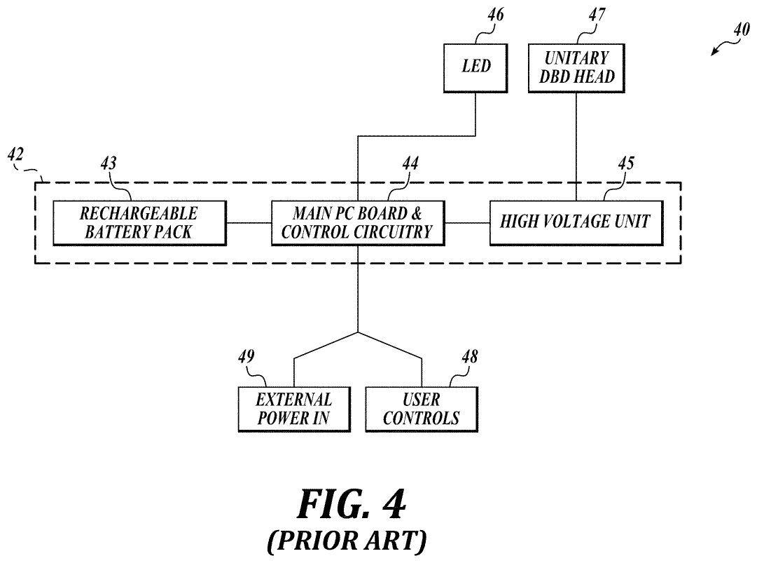

### DETAILED DESCRIPTION

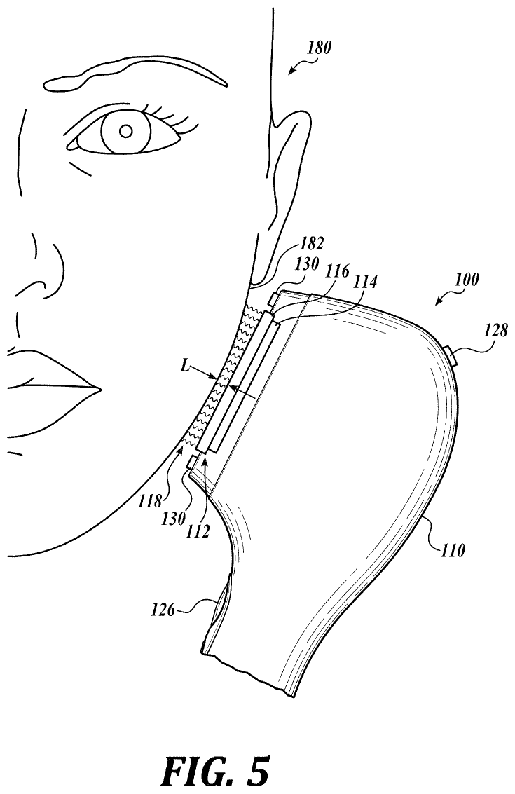

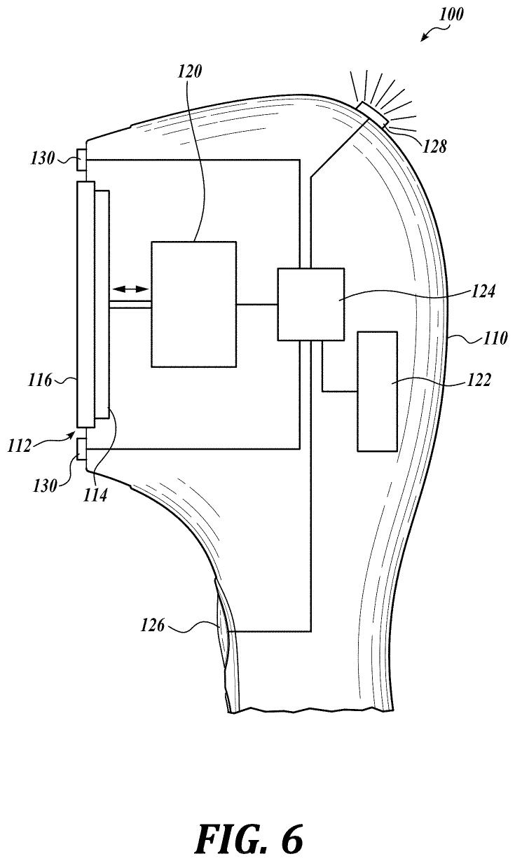

FIGS. 5 and 6 show a representative embodiment of a cold plasma device 100 for treating a biological surface 182 of a user 180 according to the present disclosure. The cold plasma device 100 includes a housing 110 in which components of the device are disposed. In an embodiment, the housing 110 is sized such that the device is a handheld device. In an embodiment, some components are disposed within the housing, and other components are disposed within a base unit that is operably coupled to the housing via a wired or wireless connection.

A cold plasma generator 112 is mounted to the housing 110 for reciprocating movement relative to the housing. The cold plasma generator 112 includes an electrode 114 coupled to a first side of a dielectric barrier 116. The cold plasma generator 112 is positioned so that a second side of the dielectric barrier 116, i.e., the side that faces away from the electrode 114, faces the biological surface 182 when the cold plasma device 100 is being used.

In an embodiment, the cold plasma generator 112 is slidably mounted to the housing 110. In an embodiment, the cold plasma generator 112 is rotatably mounted at one end to the housing 110, wherein the opposite end is moveable to rotate the center of the cold plasma generator toward or away from the biological surface being treated. In an embodiment, the cold plasma generator 112 is mounted to the housing 110 by one or more links or linkages to provide for movement of the cold plasma generator relative to the housing. The present disclosure is not limited to any particular mounting configuration. In this regard, it will be appreciated that the cold plasma generator 112 can be mounted to the housing directly or indirectly by a number of suitable configurations, and such configurations should be considered within the scope of the present disclosure.

An actuator 120 is mounted within the housing 110 and is coupled to the cold plasma generator 112 to selectively position the cold plasma generator. The actuator 120 is operably connected to a controller 124 that selectively positions the cold plasma generator 112 by controlling the actuator. The controller 124 is also operably connected to a power source 122 that supplies power to the cold plasma generator 112 and other components of the cold plasma device 110 that require power. In some embodiments, the cold plasma device includes one or more of a user control 126, a display 128, and at least one sensor 130 operably connected to the controller 124.

In an embodiment, the actuator 120 is a servo motor. In an embodiment, the actuator 120 is stepper motor. In an embodiment, the actuator 120 is a rotary actuator. In an embodiment, the actuator 120 is a linear actuator. In an embodiment, the actuator 120 is coupled to cold plasma generator 124 by one or more links. In an embodiment, the actuator 120 is coupled to cold plasma generator 124 by one or more gears or a rack and pinion configuration. In an embodiment, the actuator 120 positions the cold plasma generator 124 by rotating a cam against a bearing surface or follower that is coupled to the cold plasma generator. It will be appreciated that the actuator 120 can be any suitable actuator associated with the cold plasma generator 112 by any suitable configuration to selectively position the cold plasma generator 112 relative to the housing 110, and any such actuators and configurations should be considered within the scope of the present disclosure.

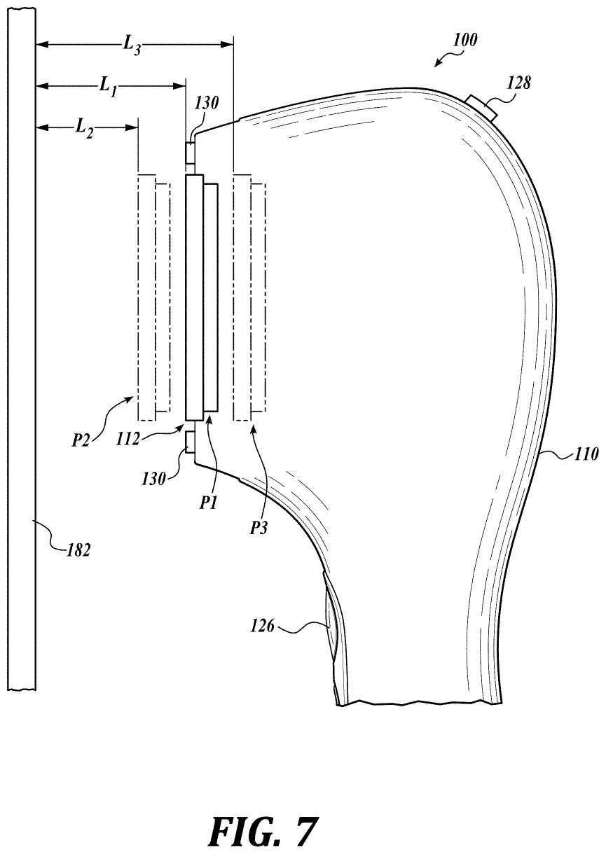

Referring now to FIG. 7, the actuator 120 is configured to position the cold plasma generator 112 relative to the housing 110. In a first position P1, the cold plasma generator 112 is at a neutral baseline position. The actuator 120 is configured to move the cold plasma generator 112 in a first direction toward a second position P2, which is closer to biological surface 182 being treated, and toward a third position P3, which is further away from the biological surface being treated. In this manner, for a given distance between the housing 110 and the biological surface 182, the actuator 120 is capable of changing the distance L between cold plasma generator 112 and the biological surface 182 from a baseline distance L1 at position P1 to a distance between the distance L2 at position P2 and the distance L3 at position P3. Further, as the housing 110 moves relative to the biological surface 182, the cold plasma generator 112 can by moved relative to the housing so that the distance between the biological surface and the cold plasma generator is maintained.

In some embodiments, the actuator 120 is configured to position the cold plasma generator 112 in multiple additional positions between the positions P2 and P3. In some embodiments, the actuator 120 is configured to position the cold plasma generator 112 in just two positions, e.g., positions P2 and P3.

Still referring to FIG. 7, the cold plasma device 100 the one or more sensors 130 are positioned to sense characteristics related to the biological surface 182. In an embodiment, at least one sensor 130 senses a distance between the sensor and the biological surface 182 and sends a signal to the controller 124 corresponding to the sensed distance. In an embodiment, at least one sensor 130 captures digital images the biological surface 182. In an embodiment, the digital image is a photograph. The controller 124 is programmed to analyze the digital images to identify features of the biological surface 182. In an embodiment, the features include but are not limited to blackheads, pores, and/or scars.

In operation, a user 180 holds the housing 110 of the cold plasma device 100 proximate to an area of the biological surface 182 to be treated; however, it is difficult for a user to hold the device with the precision required to optimize the space between the biological surface and the cold plasma generator 112 for maximum efficacy. Some known devices include offset features that contact the biological surface 182 to provide a predetermined space between the biological surface and the cold plasma generator 112; however, the pliable nature of biological surfaces allow for variation in the space between the biological surface and the cold plasma generator depending upon how much pressure a user 180 applies to engage the offset features with the biological surface. That is, applying more pressure to the device may result in a smaller than desired space between the biological surface 182 and the cold plasma generator 112.

### Open Loop Control

In some embodiments the controller 124 is programmed to operate the cold plasma device 100 as an open loop system. In an embodiment, a user sets a particular setting using the user control 126. In one embodiment, the setting is an input corresponding to an optimal distance between the biological surface 182 and the cold plasma generator 112. In an embodiment, the setting is an input corresponding to the biological surface 182 of a particular body part, such as a forehead, a cheek, a hand, or any other body part that may be treated by the cold plasma device 100. In an embodiment, the setting is a plasma concentration to be generated by the cold plasma generator 112.

With the input set by the user, the cold plasma device 100 is activated. In some embodiments, the controller controls the actuator to maintain the cold plasma generator 112 at a predetermined distance from the biological surface 182, even as the distance between the housing 110 and the biological surface changes. In an embodiment, the controller provides an alert to the user when the cold plasma generator 112 is beyond a predetermined distance from the biological surface 182. In an embodiment, the signal is a visual signal indicated on the display 128. In an embodiment, the signal is an audible signal or a haptic signal or any other suitable type of signal or combination of signals.

In an embodiment, the controller 124 controls the voltage provided to the electrode 114 of the cold plasma generator 112 according to the user input setting. The concentration of plasma generated by the cold plasma generator 112 increases and decreases with the applied voltage. Accordingly, in this manner the controller increases or decreases the plasma concentration as required by the user input setting. In an embodiment, the controller 124 controls both (1) the distance between the cold plasma generator 112 and the biological surface, and (2) the plasma concentration according to the user input setting.

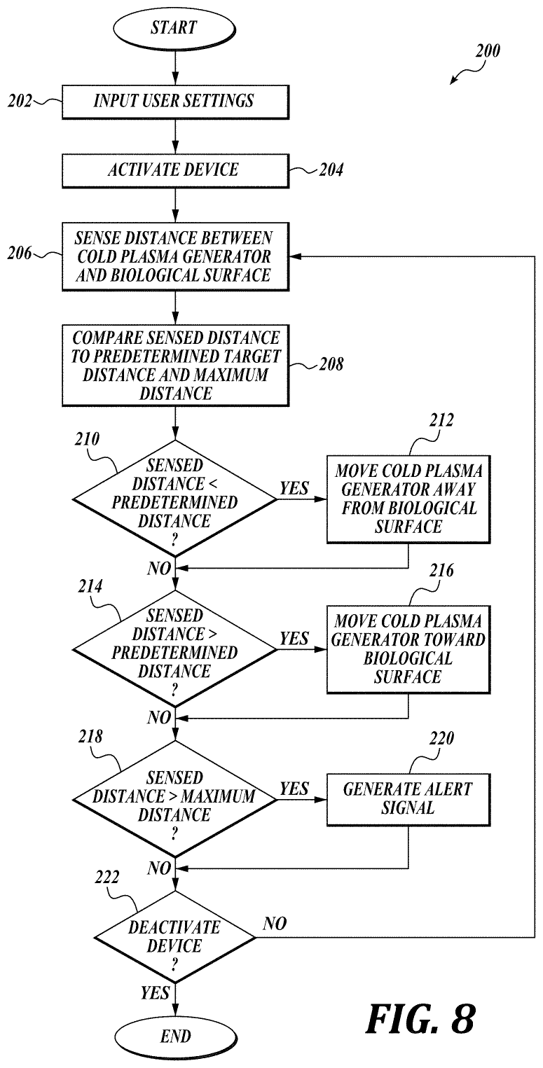

FIG. 8 shows an embodiment of a method 200 for using a cold plasma device 100 to treat a biological surface according to the present disclosure. The method 200 starts by proceeding to block 202, in which user settings are input. The method 200 proceeds to block 204, in which the device is activated.

In block 206, the distance between the cold plasma generator 112 and the biological surface 182 is sensed. In block 208, the sensed distance is compared to (1) a predetermined target distance and (2) a predetermined maximum distance based on the user settings.

The method 200 proceeds to block 210. If the sensed distance is less than the predetermined distance, the method 200 proceeds to step 212, and the actuator 120 moves the cold plasma generator 112 away from the biological surface 182. The method 200 then proceeds to block 214. If the sensed distance is greater than the predetermined distance in block 210, the method 200 proceeds directly from block 210 to block 214.

In block 218, if the sensed distance is greater than the predetermined distance, the method 200 proceeds to step 216, and the actuator 120 moves the cold plasma generator 112 toward the biological surface 182. The method 200 then proceeds to block 218. If the sensed distance is less than the predetermined distance in block 214, the method 200 proceeds directly from block 214 to block 218.

In block 218, if the sensed distance is greater than a predetermined maximum distance, the method 200 proceeds to block 220, and the controller controls the display 128 to generate an alert signal. The method 200 then proceeds to block 222. If the sensed distance is less than the predetermined maximum distance, the method 200 proceeds directly from block 218 to block 222.

In block 222, if the cold plasma device 100 has been deactivated, then the method 200 proceeds to an end block and terminates. If the cold plasma device 100 has not been deactivated, then the method 200 proceeds back to block 206.

### Closed Loop Control

In some embodiments the controller 124 is programmed to operate the cold plasma device 100 as a closed loop system. In an embodiment, a user sets a particular setting using the user control 126. In one embodiment, the setting is an input corresponding to a particular treatment or a treatment of the biological surface 182 of a particular body part, such as a forehead, a cheek, a hand, or any other body part that may be treated by the cold plasma device 100.

With the input set by the user, the cold plasma device 100 is activated. In some embodiments, sensor 130 senses features of the biological surface 182. In an embodiment, the feature is one or more of a pore, a whitehead, a scar, or any other feature or combination of features. In an embodiment, the controller 124 controls the actuator 120 to maintain the cold plasma generator 112 at a predetermined distance from the biological surface 182, wherein the predetermined distance corresponds to the user settings and a sensed feature of the biological surface. In an embodiment, the controller 124 controls the voltage applied to the electrode 114 so that the cold plasma generator 112 generates a predetermined plasma concentration, wherein the predetermined plasma concentration corresponds to the user settings and a sensed feature of the biological surface.

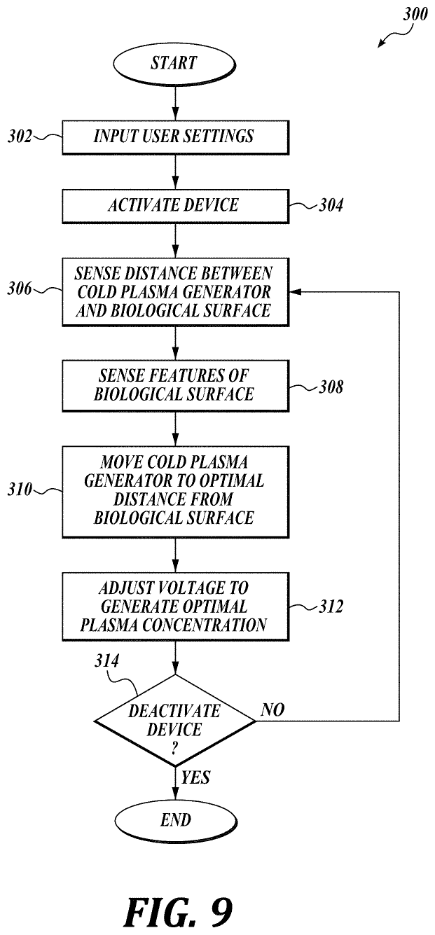

FIG. 9 shows an embodiment of a method 300 for using a cold plasma device 100 to treat a biological surface according to the present disclosure. The method 300 starts by proceeding to block 302, in which user settings are input. The method 300 proceeds to block 304, in which the device is activated.

In block 306, the distance between the cold plasma generator 112 and the biological surface 182 is sensed. The method 300 then proceeds to block 308, wherein features of the biological surface 182 are sensed.

In block 308, the controller 124 controls the actuator 120 to position the cold plasma generator 112 at an optimal distance from the biological surface 182, wherein the optimal distance is based at least in part on the sensed feature. The method next proceeds to block 312, in which the controller 124 adjusts the voltage being applied to the electrode 114 of the cold plasma generator 112 so that the cold plasma generator generates a plasma concentration based at least in part on the sensed features of the biological surface 182.

In block 314, if the cold plasma device 100 has been deactivated, then the method 300 proceeds to an end block and terminates. If the cold plasma device 100 has not been deactivated, then the method 300 proceeds back to block 306.

The detailed description set forth above in connection with the appended drawings where like numerals reference like elements, are intended as a description of various embodiments of the present disclosure and are not intended to represent the only embodiments. Each embodiment described in this disclosure is provided merely as an example or illustration and should not be construed as preferred or advantageous over other embodiments. The illustrative examples provided herein are not intended to the exhaustive or to limit the disclosure to the precise forms disclosed. Similarly, any steps described herein may be interchangeable with other steps, or combinations of steps, in order to achieve the same or substantially similar result. Moreover, some of the method steps can be carried serially or in parallel, or in any order unless specifically expressed or understood in the context of other method steps.

In the foregoing description, specific details are set forth to provide a thorough understanding of exemplary embodiments of the present disclosure. It will be apparent to one skilled in the art, however, that the embodiments disclosed herein may be practiced without embodying all of the specific details. In some instances, well-known process steps have not been described in detail in order not to unnecessarily obscure various aspects of the present disclosure. Further, it will be appreciated that embodiments of the present disclosure may employ any combination of features described herein.

The present application may also reference quantities and numbers. Unless specifically stated, such quantities and numbers are not to be considered restrictive, but exemplary of the possible quantities or numbers associated with the present application. Also, in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value.

Throughout this specification, terms of art may be used. These terms are to take on their ordinary meaning in the art from which they come, unless specifically defined herein or the context of their use would clearly suggest otherwise.

The principles, representative embodiments, and modes of operation of the present disclosure have been described in the foregoing description. However, aspects of the present disclosure, which are intended to be protected, are not to be construed as limited to the particular embodiments disclosed. Further, the embodiments described herein are to be regarded as illustrative rather than restrictive. It will be appreciated that variations and changes may be made by others, and equivalents employed, without departing from the spirit of the present disclosure. Accordingly, it is expressly intended that all such variations, changes, and equivalents fall within the spirit and scope of the present disclosure as claimed.

1. A cold plasma device for treating a region of a biological surface, the device comprising:
a cold plasma generator having an electrode and a dielectric barrier, the dielectric barrier having a first side that faces the electrode and a second side that faces away from the electrode;
an actuator configured to selectively reciprocate the cold plasma generator between a first position and a second position; and
a controller operably coupled to the actuator and programmed to control the actuator to selectively position the cold plasma generator relative to the biological surface.

2. The cold plasma device of claim 1, further comprising a sensor configured to sense a distance between the cold plasma generator and the biological surface.

3. The cold plasma device of claim 2, wherein the controller is configured to control the actuator to maintain the cold plasma generator at a predetermined distance from the biological surface.

4. The cold plasma device of claim 3, wherein the controller is configured to receive a signal from the sensor, the controller being programmed to control the actuator according to the received signal.

5. The cold plasma device of claim 4, wherein the sensor senses a distance between the cold plasma generator and the biological surface.

6. The cold plasma device of claim 4, wherein the sensor senses a feature on the biological surface, the controller being programmed to control the actuator to position the cold plasma generator according to the sensed feature.

7. The cold plasma device of claim 1, wherein the actuator is a linear actuator.

8. The cold plasma device of claim 1, wherein the actuator is a rotary actuator.

9. The cold plasma device of claim 1, further comprising a housing, wherein the cold plasma generator is moveably mounted to the housing.

10. The cold plasma device of claim 1, wherein the cold plasma generator is slidably mounted to the housing

11. A method of treating a biological surface with a cold plasma generator located a distance from the biological surface and generating a plasma concentration, the method comprising the steps of:
sensing the distance between the cold plasma generator and the biological surface;
comparing the sensed distance to a predetermined distance; and
moving the cold plasma generator relative to the biological surface according to the comparison of the sensed distance and the predetermined distance.

12. The method of claim 11, wherein the predetermined distance is a target distance, and the step of moving the cold plasma generator moves the cold plasma generator such that the sensed distance approaches the predetermined distance.

13. The method of claim 11, further comprising the step of sensing a feature on the biological surface.

14. The method of claim 13, further comprising the step of adjusting the plasma concentration generated by the cold plasma generator according to the sensed feature.

15. The method of claim 14, further comprising the step of adjusting the plasma concentration generated by the cold plasma generator according to the sensed feature.

16. The method of claim 14, further comprising the step of adjusting the distance between the cold plasma generator and the biological surface according to the sensed feature.
